(19) United States
(12) Patent Application Publication
(10) Pub. No.: US 2025/0009645 A1
(43) Pub. Date: Jan. 9, 2025

(54) ULTRA LONG ACTING PHARMACEUTICAL COMPOSITION COMPRISING INSULIN

(71) Applicants: Yong Soo JUN, Uijeongbu-si, Gyeonggi-do (KR); UNDBIO CO., LTD., Uijeongbu-si, Gyeonggi-do (KR)

(72) Inventors: Yong Soo JUN, Uijeongbu-si (KR); Hyuk Jin KWON, Hwaseong-si (KR); Maharaj Kishen SAHIB, Maharashtra (IN); Soma Sekhar PENUMAJJI, Telangana State (IN); Hemant Ramesh PATIL, Maharashtra (IN); Satyashila Babaji AVHAD, Maharashtra (IN); Ji Hyeong PARK, Suwon-Si (KR); Ga Vin KIM, Gangneung-si (KR)

(73) Assignees: Yong Soo JUN, Uijeongbu-si, Gyeonggi-do (KR); UNDBIO CO., LTD., Uijeongbu-si, Gyeonggi-do (KR)

(21) Appl. No.: 18/711,430

(22) PCT Filed: Nov. 23, 2022
(86) PCT No.: PCT/KR2022/018645

§ 371 Date: May 17, 2024

(30) Foreign Application Priority Data
Nov. 24, 2021 (WO) PCT/KR2021/017429

(51) Int. Cl. A61K 9/00; A61K 38/28; A61K 47/02; A61K 47/10; A61K 47/26; A61K 47/42

(52) U.S. Cl. CPC A61K 9/0019; A61K 38/28; A61K 47/02; A61K 47/10; A61K 47/26; A61K 47/42

(57) ABSTRACT

The present invention discloses a stable ultra long acting injectable solution comprising Insulin glargine, collagen and one or more pharmaceutically acceptable excipients or carrier at pH 2-5. The present invention also discloses the process for preparing the said composition and its use in treatment of metabolic disorders including diabetes mellitus.

### TECHNICAL FIELD

The present disclosure broadly lies in the field of biopharmaceuticals, particularly, to Ultra long acting insulin glargine formulation. Specifically, the present invention relates to a stable injectable solution comprising Insulin glargine, collagen and one or more pharmaceutically acceptable excipients or carrier thereof composition. The present invention also discloses the process for preparing the said composition and its use in the treatment of metabolic disorders including diabetes mellitus.

### BACKGROUND ART

Diabetes is a serious medical condition characterized by the body's inability or deficiency to metabolize glucose. This disease affects around 9.3 percent of the global adult population in 2019 and by the year 2045 this number is expected to rise to almost 11 percent (https://www.statista.com/statistics/271464/percentage-of-diabetics worldwide/).

Diabetes is the leading cause of death globally. There are two major types of diabetes mellitus: Type 1 diabetes, caused by insufficient secretion of insulin due to the damage of pancreatic beta cells, which requires frequent administration of exogenous insulin to Sustain life; and

Type 2 diabetes, often caused by inadequate endogenous insulin to control glucose levels, which is currently managed through dietary modifications, exercise, medication, or through insulin injections in about 20% of the cases.

In both types of diabetes, hypoglycemia frequently results from the use of insulin, owing to a very poor approximation of normal physiological insulin secretion that is tightly modulated by glucose levels. In order to maintain blood glucose levels within the normal range, diabetic patients have to administer insulin periodically.

Insulin injections are prescribed to patients suffering from diabetes. Insulin is a natural hormone, which controls the level of sugar glucose in the blood. In healthy people, insulin is released in the blood by the pancreas as the concentration of blood glucose rises. Increased blood glucose levels, occur after meals and are rapidly compensated by a corresponding increase in insulin secretion. Insulin plays major role in converting excess blood glucose into glycogen and storing it in the liver.

Since the introduction of insulin in the 1920's, continuous efforts have been made to improve the treatment of diabetes mellitus. To help avoid extreme glycaemia levels, diabetic patients often practice multiple injection therapy, whereby insulin is administered with each meal.

Insulin is a polypeptide of 51 amino acids, which are divided into 2 amino acid chains: the A chain having 21 amino acids and the B chain having 30 amino acids. The chains are connected to one another by means of two disulfide bridges. Insulin preparations have been employed for diabetes therapy for many years.

Traditionally short acting regular Insulin formulations or its intermediate acting Insulin Protamine formulations were used for treating patients with diabetes mellitus. With time, new insulin analogues and derivatives were developed. Insulin analogues and derivatives differ from human insulin at one or more than one amino acid position and/or amino acid chain length.

A number of insulin, insulin analogues and derivatives are available in the market. The commonly used types of insulin, insulin analogues or insulin derivatives are categorized as:
Rapid-acting Insulin analogs (Bolus): For example insulin aspart (Novolog®); Insulin Lispro (Humalog®); Insulin glulisine (Aprida®), Rapid acting human insulin (Viaject®). These analogues begin to work within 5 to 15 minutes of administration and are active for 3 to 4 hours.

Short-acting insulin (Bolus): For example Regular insulin (Humulin® or Novolin®). Regular insulin starts working within 30 minutes after administration and duration of action lasts from about 5 to 8 hours.

Intermediate-acting insulin: For example as isophane insulin, Aspart protamine, and Lispro protamine. It starts working in 1 to 3 hours after administration. Its duration of action varies between 16 to 24 hours.

Long-acting insulin (Basal): For example Insulin glargine, insulin degludec and insulin detemir. These analogs start working within 1 to 2 hours and their duration of action varies from about 12 to about 24 hours.

Mixed Insulin: For example mixture of NPH and regular insulin. There are several variations with different proportions of the mixed insulins. The onset of action of these mixed preparations is about 30 minutes. The mixed insulin comprises the same type of insulin. Two different type of insulins cannot be mixed i.e. insulin Lispro cannot be mixed with insulin detemir, insulin aspart or insulin glargine. The mixed formulation of insulin Lispro can only comprise insulin Lispro regular and insulin Lispro protamine-two forms of insulin Lispro.

Insulin Glargine is one of the long acting basal insulin, which is administered once a day. Insulin glargine is formulated in an acidic solvent and it has an altered isoelectric point. Insulin glargine is isoelectric at physiological pH, it precipitates after subcutaneous injection, forming an amorphous depot. Gradual re-dissolution from this subcutaneous depot is the key retardation principle. The longer duration of action (up to 24 hours) of insulin glargine is directly related to formation of micro-precipitate particle; rate of dissociation of micro-precipitate in to monomer unites of insulin glargine and slower rate of absorption of insulin glargine from subcutaneous tissue. However, in many patients, Insulin glargine does not last a full 24 hours. The drug precipitates after injection into the subcutaneous tissue and is slowly re-dissolved and absorbed from here. It has a “peak less” action, but the drug is associated with interindividual variability, and many patients exhibit peaks in action, especially at higher doses.

A further disadvantage of insulin glargine is that, unlike isophane insulin, it cannot be mixed with soluble insulins as this result in precipitation. Thus, many patients with biphasic (mixture) insulins will be required to increase their number of daily injections and/or change to a basal-bolus injection regimen. Further, in many patients insulin resistance develops with chronic administration of basal insulin, which requires administration of higher doses of insulin. In vitro studies have demonstrated that glargine binds preferentially to insulin-like growth factor-1 (IGF-1) receptors more than to insulin receptors. This may create a mitogenic potential, especially with long-term use. Thus, there is a need for insulin glargine formulation which not only exhibits a longer duration of action but also has reduced inter-individual variability and less mitogenic potential.

Biomaterials are being widely utilized in the drug and pharmaceutical for efficient drug delivery in the body. Among the different known biopolymers, collagen has come up as one of the most attractive choice due to its excellent biocompatibility, biodegradability and weak antigencity, well-established structure, biological characteristics and to the way it interacts with the body (Friess, 1998; Lee et al., 2001).

Collagen, the most abundant protein in mammals represents the chief structural protein accounting for approximately 30% of all vertebrate body protein. More than 90% of the extracellular protein in the tendon and bone and more than 50% in the skin consist of collagen. Animal skin, mostly bovine or porcine or Achilles tendons, mostly bovine or equine currently comprise the main source of collagen.

So far, 28 types of collagen have been identified and described, most common being;
Type I: Main component of the organic part of bone.
Type II: main component of cartilage.
Type III: main component of reticular fibers.
Type IV: main component of basal lamina, the epithelium-secreted layer of the basement membrane.
Type V: mainly found in cell surfaces, hair and placenta.

There are a number of prior arts disclosing the binding affinity of insulin with collagen and pharmaceutical composition comprising the combination.

U.S. patent application No. 20130225492 A1 discloses a pharmaceutical composition, including a drug and collagen, in which the composition is satisfactory in handleability and has sustained-release properties. The sustained-release pharmaceutical composition includes: a drug; collagen; and at least one kind of sugar selected from monosaccharides, disaccharides, trisaccharide and tetrasaccharides.

U.S. Pat. No. 5,922,356 B2 discloses a sustained release formulation used for treatment or prevention of the disease, which contains a therapeutically effective substance as an active ingredient, collagen as a drug carrier and glycosaminoglycan as an additive.

JP Patent 543453 discloses a local sustained-release formulation for wound healing promotion obtained by mixing a carrier containing collagen as an essential constituent with a physiologically active substance having a wound healing promoting activity

JP Patent 56122317 discloses that when the collagen concentration is reduced, degradation of a gelated collagen containing a drug is accelerated; sustained release of the drug is also accelerated.

Yaoi et al., 1991 discloses the binding efficiency of insulin with collagen by analyzing the abilities of eight extracellular matrix proteins, fibronectin, vitronectin, laminin, collagen types I, II, III, IV and V to bind to insulin.

Manolache et al., 2016 discloses gel composites and their corresponding matrices comprising collagen gel, zinc oxide and insulin glargine at pH 7.4.

U.S. Pat. No. 6,468,959 discloses Dry pellets containing peptide pharmaceuticals, in oral, buccal, sublingual or nasally administrable dosage form comprising at least one peptide pharmaceutical having a given net charge dispersed in a matrix which comprises a member selected from the group consisting of gelatin, fractionated gelatins, collagen hydrolysates, crosslinked gelatins and mixtures thereof, said member or members of said group of hydrophilic molecules carrying a sufficient contrary net charge to form a psuedocoacervate with said peptide pharmaceutical.

U.S. patent application No. 20140213963 discloses a biocompatible insulin delivery device comprising a reservoir for insulin, a glucose-responsive plug that seals the reservoir, and optionally comprising a protective microporous membrane covering the exposed surface of the glucose-responsive plug and further sealing the reservoir, wherein the plug comprises a polymeric matrix having an inorganic component and a stimulus-responsive component adapted to alter the porosity of the plug in response to a stimulus and wherein the plug functions to release insulin from the reservoir in response to a hyperglycemic glucose concentration and to prevent insulin release from the reservoir in response to a hypoglycemic glucose concentration.

The composition comprising insulin and collagen are known in the prior arts. The compositions of prior arts are gel formulations, pellets or implants. The administration of insulin in gel or as implants requires specialized syringes or devices, which reduces the case of administration. For diabetics, case of administration matters as they require repeated administration of insulin. Administration of gels or implants causes pain at site of injection and often swelling occurs, which further causes inconvenience to patient leading to non-compliance. Also, these specialized syringes or device increases the total cost of the product, making it unaffordable for many patients. Oral administration of insulin as pellets is associated with large variability in absorption. The pharmacokinetic response from oral route in case of insulin is often unpredictable.

### DISCLOSURE OF INVENTION

### Technical Problem

Thus, there is a need of insulin glargine and collagen formulation which not only is easy to administer using standard pens and syringes but also has increased bioavailability, longer duration of action and reduced immunogenicity compared to the marketed insulin glargine compositions at same dose.

None of the prior arts disclose an injectable solution comprising insulin glargine, collagen and one or more pharmaceutically acceptable excipients having pH 2-5, wherein the pharmaceutical composition exhibits longer duration of action and increased bioavailability compared to the marketed compositions of insulin glargine at same concentration.

### Solution to Problem

The term “insulin(s)” used herein includes mammalian insulin, insulin analogues or derivatives.

The term “Insulin analogs or derivatives” used in the present invention includes analogs or derivatives of naturally occurring insulins, namely human insulin or animal insulins, brought about by modifying the structure of human insulin molecule, which results in altered physicochemical, pharmacokinetic and pharmacodynamic properties.

One of the aspects of the present invention provides a stable ultra long acting injectable pharmaceutical composition comprising an insulin analogue or derivative having an isoelectric point between 5 and 8.5, or a physiologically tolerated salt thereof, collagen and one or more pharmaceutically acceptable excipients or carrier, wherein, the pharmaceutical composition has pH between 2-5; the pharmaceutical composition is a solution when administered and forms a depot upon administration at physiological pH; and the pharmaceutical composition exhibits increased bioavailability and longer duration of action compared to the marketed composition comprising the said insulin analogue or derivative having an isoelectric point between 5 and 8.5 at the same dose.

One of the aspects of the present invention provides a stable ultra long acting injectable pharmaceutical composition comprising insulin glargine, or a physiologically tolerated salt thereof, collagen and one or more pharmaceutically acceptable excipients or carrier, wherein the pharmaceutical composition has pH between 2-5; the pharmaceutical composition is a solution when administered and forms a depot upon administration at physiological pH; and the pharmaceutical composition exhibits increased bioavailability and longer duration of action compared to the marketed composition comprising insulin glargine at same dose.

One of the aspect of the present invention provides a stable ultra long acting injectable pharmaceutical composition comprising insulin glargine, or a physiologically tolerated salt thereof, collagen and one or more pharmaceutically acceptable excipients or carrier, wherein the pharmaceutical composition has pH between 2-5; the pharmaceutical composition is a solution when administered and forms a depot upon administration at physiological pH; and the pharmaceutical composition exhibits increased bioavailability and duration of action of more than 24 h.

One of the aspect of the present invention provides a stable ultra long acting injectable pharmaceutical composition comprising insulin glargine, or a physiologically tolerated salt thereof, human collagen and one or more pharmaceutically acceptable excipients or carrier, wherein the pharmaceutical composition has pH between 2-5; the pharmaceutical composition is a solution when administered and forms a depot upon administration at physiological pH; and the pharmaceutical composition exhibits increased bioavailability and longer duration of action compared to the marketed composition comprising insulin glargine at same dose.

One of the aspects of the present invention provides a stable ultra long acting injectable pharmaceutical composition comprising an insulin analogue or derivative having an isoelectric point between 5 and 8.5, or a physiologically tolerated salt thereof, collagen and one or more pharmaceutically acceptable excipients or carrier, wherein, the pharmaceutical composition has pH between 2-5; the pharmaceutical composition is a solution when administered and forms a depot upon administration at physiological pH; the pharmaceutical composition exhibits increased bioavailability and longer duration of action compared to the marketed composition comprising the said insulin analogue or derivative having an isoelectric point between 5 and 8.5 at same dose; and wherein the pharmaceutically acceptable excipients or carrier is selected from the group consisting of one stabilizers, isotonic agents, surfactants, buffers, preservatives, antioxidants, acidifying agents, pH modifying agents, organic solvents, aqueous solvents, solubilizing agents and combination thereof.

One of the aspect of present invention provides a method for treating Type I and Type II Diabetes Mellitus in a patient comprising administering a stable ultra long acting injectable pharmaceutical composition comprising an insulin analogue or derivative having an isoelectric point between 5 and 8.5, or a physiologically tolerated salt thereof, collagen and one or more pharmaceutically acceptable excipients or carrier, wherein, the pharmaceutical composition has pH between 2-5; the pharmaceutical composition is a solution when administered and forms a depot upon administration at physiological pH; and the pharmaceutical composition exhibits increased bioavailability and longer duration of action compared to the marketed composition comprising the said insulin analogue or derivative having an isoelectric point between 5 and 8.5 at same dose, wherein the pharmaceutical composition is administered 2 to 3 times a week.

One of the aspects of present invention provides a process for preparing a stable ultra long acting injectable solution comprising an insulin analogue or derivative having an isoelectric point between 5 and 8.5, or a physiologically tolerated salt thereof, collagen and one or more pharmaceutically acceptable excipients or carrier having pH between 2-5, wherein the process comprises
a) dissolving accurately weighed zinc-containing crystals of insulin glargine in water for injection with the help of few μL of 1M HCl;
b) preparing buffer having pH 2-5;
c) dissolving isotonic agent in buffer;
d) diluting concentrated insulin glargine of step A with the buffer of step b)
e) adding collagen the solution of step d) with constant stirring;
f) adjusting the final pH of the solution between 2-5.

### Advantageous Effects of Invention

The present invention discloses a stable ultra long acting injectable solution comprising Insulin glargine, collagen and one or more excipients thereof at pH 2-5.

The present invention also discloses the process for preparing the said composition and its use in treatment of metabolic disorders including diabetes mellitus.

### BRIEF DESCRIPTION OF DRAWINGS

FIG. 1 shows Standard Curve of Insulin Glargine in presence of Collagen.

FIG. 2 depicts Simulation of plasma insulin concentration with once daily Gla-100 or Gla-300 as per prior art. Reference: Modelling of Subcutaneous Absorption of Long-Acting Insulin Glargine in Type 1 Diabetes Michele et al: IEE Trans Biomed Eng 202; 67(2): 624-631.

FIG. 3: Proposed In-vitro model of Insulin Glargine Release.

### MODE FOR THE INVENTION

While working on different possible formulations to prepare a pharmaceutical composition comprising insulin glargine, which not only exhibits a 24 h+ duration of action but is also easy to administer, more bioavailable and less immunogenic, inventors of the present invention observed that human type I collagen, when mixed with insulin glargine at acidic pH can significantly reduce the formation of M-1 and M-2 (on exposure to proteases in physiological saline mimicking the interstitial fluid composition), the soluble metabolites of insulin glargine at physiological pH. These metabolites of insulin Glargine are responsible for its hypoglycemic activity and hence any formulation of the drug that can reduce formation of these metabolites will have direct correlation with extent of sustained release of the drug on the one hand and extent of flatness of the in vitro M-1, M-2 formation kinetics will physiologically/pharmacologically translate into improved pharmacological activity with longer half-life of the active drug in circulation as long as the depot is present at site of injection.

The compositions of present invention are acidic and when administered in-vivo forms a depot at physiological pH. Inventors of the present invention observed that at the site of injection (physiological pH) Type I collagen molecule, which is a triple helix protein, entraps insulin, thereby forming an additional barrier to already crystallized insulin glargine at site of injection at physiological pH 7.4. by inhibition of proteases in interstitial fluid of extracellular matrix at injection site. Two compartment model for insulin glargine absorption into systemic circulation has been proposed. First compartment for dissolution of precipitate to hexamer and then hexamer to monomer second compartment being blood after transport through blood capillary pores in subcutanenous tissue. Thus, the release of the entrapped insulin is further controlled by this additional barrier of collagen at physiological pH 7.4. Further, collagen inhibits proteases which degrade insulin at the site of injection. This inhibition of proteases increases the bioavailability of insulin in blood. Collagen acts synergistically with insulin and helps in controlling the blood sugar level. Thus, the pharmaceutical composition of present invention has better bioavailability compared to market composition containing insulin glargine at same concentration.

The pharmaceutical compositions of present invention are solution as both insulin glargine and collagen are soluble at acidic pH. The solution of present invention are easy to administer with conventional syringe or pens and do not require any specialized syringe or device to administer. Ease of administration leads to patient compliance. Further, as the pharmaceutical compositions of present invention are easily administered through conventional pens and syringes, they are cost effective and does not create any additional burden on patients.

As used herein the term “pharmaceutical composition” means a mixture containing a therapeutic compound to be administered to a mammal, e.g., a human, in order to prevent, treat or control a particular disease or condition affecting the mammal.

The “collagen” used herein means fibrous protein that is constituent of bone, cartilage, tendon, and other connective tissue. The “collagen” as used herein include, but not limited to Collagen Type I, II, III, IV, V, human collagen, collagen which have been processed or otherwise modified.

The term “buffers” used herein means a solution containing either a weak acid and its salt or a weak base and its salt, which is resistant to changes in pH. The “buffers” as used herein include, but are not limited to, phosphate, acetate, citrate, arginine, glycylglycine or TRIS (i.e. 2-amino-2-hydroxymethyl-1,3-propanediol) buffer and corresponding salts and combination thereof.

By “preservatives” as used herein refers to the compound that can be used to prevent the growth of fungi and other microorganisms. The “preservatives” as used herein include, but are not limited to, benzoic acid, butyl paraben, ethyl paraben, methyl paraben, propyl paraben, sodium benzoate, sodium propionate, benzalkonium chloride, benzethonium chloride, benzyl alcohol, cetypyridinium chloride, chlorobutanol, phenol, phenyl ethyl alcohol, 2-Penoxyethanol, Phenyl mercuric nitrate, Thimerosal, metacresol and combinations thereof.

By “isotonic agents” as used herein refers to a compound that is physiologically tolerated and imparts a suitable tonicity to a formulation to prevent the net flow of water across cell membranes that are in contact with the formulation. The “isotonic agents” is a compound, such as glycerin, are commonly used for such purposes at known concentrations. Other possible tonicity controlling agents include salts, e.g., sodium chloride, dextrose, lactose and combination thereof.

By “pH adjusting agent” as used herein refers to a combination of acid and alkali. The “pH adjusting agents” as used herein can be selected form the group comprising of o-phosphoric acid, citric acid, acetic acid, succinic acid, lactic acid, gluconic acid, tartaric acid, 1,2,3,4-butane tetracarboxylic acid, fumaric acid or malic acid. Alkali is selected form the group comprising of sodium hydroxide, potassium hydroxide, sodium hydroxide, ammonium hydroxide, magnesium oxide, calcium hydroxide, calcium carbonate, magnesium carbonate, magnesium aluminum silicates, diethanolamine, monoethanolamine, sodium carbonate, sodium bicarbonate or triethanolamine and combination thereof.

By “solubilizing agents” as used herein refers to a material able to solubilize or partially solubilize the therapeutic compound and/or polymer. Suitable “solubilizing agents” as used herein include, but are not limited to, include wetting agents such as polysorbates and poloxamers, non-ionic and ionic surfactants, food acids and bases (e.g. sodium bicarbonate), polyhydric alcohols, alcohols and combination thereof.

By “acidifying agents” as used herein refers to a chemical species that donates protons or hydrogen ions and/or accepts electrons. Suitable acidifying agents includes but not limited to, formic acid, ascorbic acid, aspartic acid, benzene sulphonic acid, benzoic acid, hydrochloric acid, sulphuric acid, phosphoric acid, nitric acid, tartaric acid, diatrizoic acid, glutamic acid, lactic acid, maleic acid, succinic acid, acetic acid, citric acid or anhydrous citric acid and combination thereof, including such agents in particulate solid form.

Suitable “organic solvents” as used herein includes, but not limited to, are selected from the group comprising of N-methylpyrrolidone (NMP), dicholoromethane, dimethylformamide (DMF), dimethylacetamide (DMAC), acetonitrile, tetrahydrofuran, dioxan, methanol, ethanol, isopropanol, tert-butanol and combination thereof.

Suitable “aqueous solvents” as used herein includes water, water for injection, water and alcohol mixture.

An “surfactants” as used herein include, but are not limited to, partial and fatty acid esters and ethers of polyhydric alcohols such as of glycerol, sorbitol and the like (Span®, Tween®, in particular Tween® 20 and Tween®80, Myrj®, Brij®, Cremophore® or poloxamers, Pluronics® and Tetronics®), polysorbates (Tween™), sodium dodecyl sulfate (sodium lauryl sulfate), lauryl dimethyl amine oxide, cetyltrimethylammonium bromide (CTAB), polyethoxylated alcohols polyoxyethylene sorbitan, Octoxynol (Triton X100™), N,N-dimethyldodecylamine-N-oxide, hexadecyltrimethylammonium bromide (HTAB), polyoxyl 10 lauryl ether, Brij 721™, bile salts (sodium deoxycholate, sodium cholate), polyoxyl castor oil (Cremophor™), nonylphenol ethoxylate (Tergitol™), cyclodextrins, lecithin, methylbenzethonium chloride (Hyamine™) and combination thereof.

An “antioxidants” as used herein can be selected from the group comprising of ascorbate (sodium/acid), bisulite sodium, butylated hydroxy anisole (bha), butylated hydroxy toluene (bht), cystein/cysteinate hcl, dithionite sodium (Na hydrosulite, Na sulfoxylate), gentisic acid, gentisic acid ethanolamine, glutamate monosodium, glutathione, formaldehyde sulfoxylate sodium, metabisulite potassium, metabisulite sodium, methionine, monothioglycerol (thioglycerol), propyl gallate, sulfite sodium, tocopherol alpha, alpha tocopherol hydrogen succinate, thioglycolate sodium and combination thereof.

The “one or more stabilizers” as used herein include, but are not limited to surfactants, antioxidants, preservatives, solubilizing agents, esterase inhibitors and combination thereof. The one or more stabilizers are selected from the group consisting of partial and fatty acid esters and ethers of polyhydric alcohols such as of glycerol, sorbitol and the like (Span®, Tween®, in particular Tween® 20 and Tween®80, Myrj®, Brij®, Cremophore® or poloxamers, Pluronics® and Tetronics®), polysorbates (Tween™), sodium dodecyl sulfate (sodium lauryl sulfate), lauryl dimethyl amine oxide, cetyltrimethylammonium bromide (CTAB), glycerol, polyethoxylated alcohols polyoxyethylene sorbitan, Octoxynol (Triton X100™), N,N-dimethyldodecylamine-N-oxide, hexadecyltrimethylammonium bromide (HTAB), polyoxyl 10 lauryl ether, Brij 721™, bile salts (sodium deoxycholate, sodium cholate), polyoxyl castor oil (Cremophor™), nonylphenol ethoxylate (Tergitol™), cyclodextrins, lecithin, methylbenzethonium chloride (Hyamine™), benzoic acid, Zinc as Zinc oxide, butylparaben, ethyl paraben, methyl paraben, propylparaben, sodium benzoate, sodium propionate, benzalkonium chloride, polyvinyl alcohol, benzethonium chloride, benzyl alcohol, cetypyridinium chloride, chlorobutanol, phenol, phenylethyl alcohol, 2-Penoxyethanol, Phenyl mercuric nitrate, Thimerosal, metacresol, ascorbate (sodium/acid), bisulite sodium, butylated hydroxy anisole (BHA), butylated hydroxy toluene (BHT), cysteine/cysteinate HCl, dithionite sodium (Na hydrosulite, Na sulfoxylate), gentisic acid, gentisic acid ethanolamine, glutamate monosodium, glutathione, formaldehyde sulfoxylate sodium, metabisulite potassium, metabisulite sodium, methionine, monothioglycerol (thioglycerol), propyl gallate, sulfite sodium, tocopherol alpha, alpha tocopherol hydrogen succinate, thioglycolate sodium, esterase inhibitors such as pancreatic secretary inhibitors, protease inhibitors, and serine esterase inhibitors such as aprotinin and combination thereof.

One of the aspects of the present invention provides a stable ultra long acting injectable pharmaceutical composition comprising an insulin analogue or derivative having an isoelectric point between 5 and 8.5, or a physiologically tolerated salt thereof, collagen and one or more pharmaceutically acceptable excipients or carrier, wherein, the pharmaceutical composition has pH between 2-5; the pharmaceutical composition is a solution when administered and forms a depot upon administration at physiological pH; and the pharmaceutical composition exhibits increased bioavailability and longer duration of action compared to the marketed composition comprising the said insulin analogue or derivative having an isoelectric point between 5 and 8.5 at same dose, wherein the insulin analogue or derivative having an isoelectric point between 5 and 8.5 is insulin glargine. The insulin glargine is present in concentration range of 100 IU-1000 IU/ml. In preferred embodiment of the present invention insulin glargine is present in concentration range of 100 IU-300 IU per ml.

One of the aspects of the present invention provides a stable ultra long acting injectable pharmaceutical composition. The injectable drug comprises an insulin analogue or derivative having an isoelectric point between 5 and 8.5, or a physiologically tolerated salt thereof, collagen and one or more pharmaceutically acceptable excipients or carrier, wherein, the pharmaceutical composition has pH between 2-5. the pharmaceutical composition is a solution when administered. it forms a depot upon administration as soon as it comes in contact with physiological pH and salts at the site of injection, the pharmaceutical composition exhibits increased bioavailability and longer duration of action compared to the marketed composition comprising the said insulin analogue or derivative having an isoelectric point between 5 and 8.5 at same dose. the collagen present in the composition is human collagen. Human collagen used in present invention is obtained from recombinant DNA technology. In preferred embodiment of the present invention, human collagen type I or rapid human collagen solution prepared by slow removal of salt from acidic collagen solution using EDTA and acetic acid. The collagen shall be used in concentration range of 3-10 mg/ml.

The compositions of present invention are useful for treatment of metabolic disorders. The metabolic disorders are selected from the group consisting of insulin resistance syndrome, diabetes, hyperlipidemia, fatty liver disease, cachexia, obesity, atherosclerosis and arteriosclerosis. In a preferred embodiment of the present invention, the compositions of present invention are useful in treatment of Type I and Type II diabetes.

The compositions of present invention exhibit longer duration of action, i.e. more than 24 h, when compared to the marketed composition at same dose. The compositions of present invention can be administered 1-3 times a week.

One of the aspects of present invention discloses a process for preparing a stable ultra long acting injectable solution comprising insulin glargine, or a physiologically tolerated salt thereof, collagen and one or more pharmaceutically acceptable excipients or carrier having pH 2-5, wherein the process comprises
a) dissolving accurately weighed zinc-containing crystals of insulin glargine in water for injection with the help of few μL of 1M HCl;
b) preparing buffer having pH 2-5;
c) dissolving isotonic agent in buffer;
d) diluting concentrated insulin glargine of step A with the buffer of step b)
e) adding collagen the solution of step d) with constant stirring;
f) adjusting the final pH of the solution between 2-5.

Another aspect of the present invention provides a method for modulating the release of an insulin analogue, derivative or metabolite having isoelectric point between 5.8 to 8.5 from a pharmaceutical composition, wherein the method comprises formulating insulin glargine with collagen and one or more pharmaceutically acceptable salt thereof at pH 2-5 and by precipitating the said composition at physiological pH upon administration thereby creating an additional barrier of collagen around insulin, which not only controls the release of insulin glargine precipitate to insulin glargine hexamer and then hexamer to monomer but also protects insulin from protease degradation.

Embodiments are further defined in the following examples. The following examples are for the purpose of illustration of the invention and not intended in any way to limit the scope of the invention.

### EXAMPLES

### Example 1: Preparation of Unit Composition Formula (100 IU/ml)

TABLE 1
Preparation of Insulin Glargine Composition
(100 IU/ml), 1 vial (1,000 IU/10 ml)

| S. No. | Ingredient | Quantity | |
|---|---|---|---|
| 1. | Insulin Glargine | 1,000 IU | |
| | | (36.4 mg) | |
| 2. | Rapid polymerizing recombinant human | 30-50 | mg |
| | type collagen (RPC solution) | | |
| 3. | Zinc | 250-350 | mcg |
| 4. | m-cresol | 2.5-3.0 | mg |
| 5. | Glycerol 85% | 15-25 | mg |
| 6. | Polysorbate 20 | 15-25 | mcg |

| | | |
|---|---|---|
| 7. | Water for injection | Upto 10 ml |

Composition of the ultra-long acting Glargine was achieved by first preparing Rapid Polymerizing Collagen Solution and then using it as excipient to prepare composition of the medicament.

### A. Preparation of Rapid Polymerizing Collagen (RPC) Solution:

The Solution of commercial recombinant human type 1 collagen was prepared by dissolving 2 g of the collagen in 50 ml of 1M acetic acid. Dialyzed this collagen solution against 0.5 M acetic acid and 50 mM EDTA solution and diluted it to 2 mg/ml with 0.25M acetic acid. Precipitated the collagen from the solution at its isoelectric point (pH 7). Washed the precipitate with water for injection twice. Suspended the precipitate in water for injection and dissolved the suspension by addition of minimal volume of 1 N HCl. Diluted it with water for injection to 40 mg/ml. The solution was filter sterilized by passing it through pre-sterilized 0.2 micron filter. The solution was stored at 2-8° C. in siliconized sterile glass vials.

### B. Evaluation of RPC Attribute of the Above Prepared Solution (Kinetics of Polymerization of the RPC Solution in PBS Environment)

4.5 ml aliquots of Phosphate Buffer Saline were dispensed in 5 glass test tubes. Added 0.5 ml of the above prepared, unbuffered, RPC solution and tested its polymerizing attribute and result of the test was as given below.

### Time Observation

0 Min No change
1.0 Min Opalescence starts appearing
5.0 Min Gel with clear solution interface visible
10 Min Clear Depo
20 Min Fibrils formed

This collagen starts gel formation at physiological pH and salt concentration in 1 min and complete fibrils are formed by 20 min, mimicking subcutaneous extra cellular matrix environment.

### C. Preparation of the Ultra Long Acting Insulin Glargine 100 IU/ml, 5 Vials (5,000 IU/50 ml) of Example 1

182 mg of Insulin Glargine, 5,000 IU, was suspended in 25 ml of water for injection dissolved and dissolved by addition of minimal volume of 1 M HCl. To the Insulin Glargine solution was added ZnCl₂ equivalent to 1.5 mg Zn, 13.5 mg meta-cresol, 0.1 g 85% glycerol, 0.1 mg polysorbate 20 and 200 mg RPC (5.0 ml) prepared as above. pH of this solution was adjusted 4 with a few drops of 1M HCl. Volume of this solution was made up to 50 ml with water for injection. The solution was filter sterilized by passing it through pre-sterilized 0.2 micron filter. The filtrate was dispensed in five pre-sterilized, siliconized 10 ml vials under aseptic environment.

### Example 2: Preparation of High Concentrated Unit Composition Formula (300 IU/ml)

TABLE 2
Preparation of Insulin Glargine Composition
(300 IU/ml) 1 vial (3,000 IU/10 ml)

| S. No. | Ingredient | Quantity | |
|---|---|---|---|
| 1. | Insulin Glargine | 3,000 IU | |
| | | (109.2 mg) | |
| 2. | Rapid polymerizing recombinant human | 30-50 | mg |
| | type collagen (RPC solution) | | |
| 3. | Zinc | 750-1,050 | mcg |
| 4. | m-cresol | 2.5-3.0 | mg |
| 5. | Glycerol 85% | 15-25 | mg |
| 6. | Polysorbate 20 | 15-25 | mcg |

| | | |
|---|---|---|
| 7. | Water for injection | Upto 10 ml |

Composition of the ultra-long acting Glargine was achieved by first preparing Rapid Polymerizing Collagen Solution and then using it as excipient to prepare composition of the medicament.

### A. Preparation of Rapid Polymerizing Collagen (RPC) Solution:

The RPC solution is prepared as described in Example 1

### B. Preparation of The Ultra Long Acting Insulin Glargine 300 IU/ml, 5 Vials (15,000 IU/50 ml) of Example 2

546 mg of insulin Glargine, 15,000 IU, was dissolved in acidulated water as above. To this solution was added ZnCl₂, equivalent to 4.5 mg Zn, 13.5 mg m-cresol, 0.1 g 85% glycerol, 0.1 mg polysorbate 20 and 200 mg RPC solution (5.0 ml). pH of the solution was adjusted by adding a few drops of 1 M HCl. Volume of this solution was made up to 50 ml with water for injection. The solution was filter sterilized by passing it through pre-sterilized 0.2 micron filter. The filtrate was dispensed in five presterilized, siliconized 10 ml vials under aseptic environment.

### Example 3: Preparation of Dilution Solutions for Standard Curve of Unit Composition Formula (100 IU/ml) and High Concentrated Unit Composition Formula (300 IU/ml)

### A. Preparation of Dilution Solution for Insulin Glargine Composition (100 IU/ml)

Dilution solution was prepared by was added sequentially ZnCl₂ equivalent to 1.5 mg Zn, 13.5 mg meta-cresol, 0.1 g 85% glycerol, 0.1 mg polysorbate 20 and 20 mg RPC (5.0 ml) to 25 ml water for injection. pH of this solution was adjusted to 4 with a few drops of 1M HCl. Volume of this solution was made up to 50 ml with water for injection. This dilution solution was used as placebo for diluting insulin Glargine to prepare standard curve of Insulin Glargine composition 300 IU/ml formulation.

### B. Preparation of Dilution Solution for Insulin Glargine Composition (300 IU/ml)

Dilution solution was prepared by adding sequentially ZnCl₂ equivalent to 4.5 mg Zn, 13.5 mg meta-cresol, 0.1 g 85% glycerol, 0.1 mg polysorbate 20 and 20 mg RPC (5.0 ml) to 25 ml water for injection. pH of this solution was adjusted to 4 with a few drops of 1M HCl. Volume of this solution was made up to 50 ml with water for injection. This dilution solution was used as placebo for diluting insulin Glargine to prepare standard curve of Insulin Glargine composition 300 IU/ml formulation.

### Example 4; Preparation of Test Solution and Establishment of Standard Curve for HPLC Analysis

Ultra-long acting Insulin Glargine, unit composition formula (Insulin Glargine composition) 100 IU/ml of Example 1, was used as test sample. The solution of Glargine was diluted with the dilution buffer prepared as above to different concentrations. Dilution buffer was used as blank. The diluted samples, 2 ml aliquots were placed in 5 ml 30 K_{d} cut-off Vivaspin tubes. The tubes were centrifuged for 30 min at rpm 5,000 in refrigerated centrifuge. The filtrate was used for HPLC analysis as described below.

### <HPLC Test Method>

Chromatographic system: Agilent HPLC
Mode—LC
Detector—214 nm
Column-Kromasil C18, 3.0 mm×250 mm, 4μ
Column temperature—35° C.
Flow rate—0.6 mL/min
Injection volume—5 μL

TABLE 3

| Time (min) | Solution A (%) | Solution B (%) |
|---|---|---|
| 0 | 96 | 4 |
| 20 | 83 | 17 |
| 30 | 63 | 37 |
| 33 | 96 | 4 |

Assay—94.0%-105.0% on the anhydrous basis.

### <Analysis of Test>

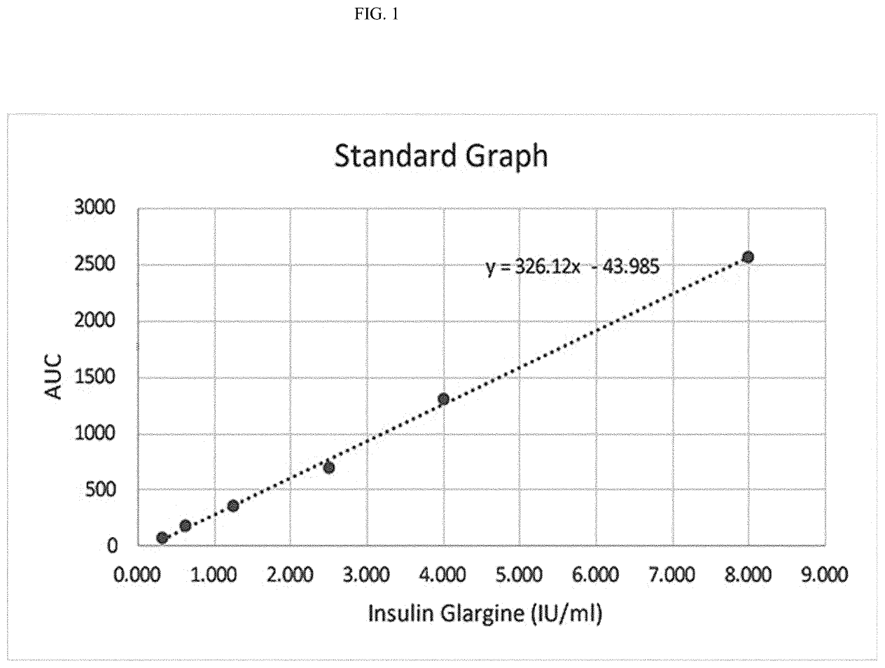

The result of analysis is tabulated below and standard curve is presented as graph in FIG. 1.

TABLE 4
Insulin Glargine Concentration dependent AUC(Area Under Curve)

| IU/mL | AUC |
|---|---|
| 8.000 | 2,569 |
| 4.000 | 1,300 |
| 2.500 | 690 |
| 1.250 | 358 |
| 0.625 | 182 |
| 0.313 | 79 |

### Example 5: In-Vitro Data Simulating Release of Insulin Glargine into Subcutaneous

Validation of in vitro model mimicking blood pK profile of in vivo insulin glargine requires understanding the in vivo model proposed for pK profile of the drug delivered by subcutaneous (“Sc”) route.

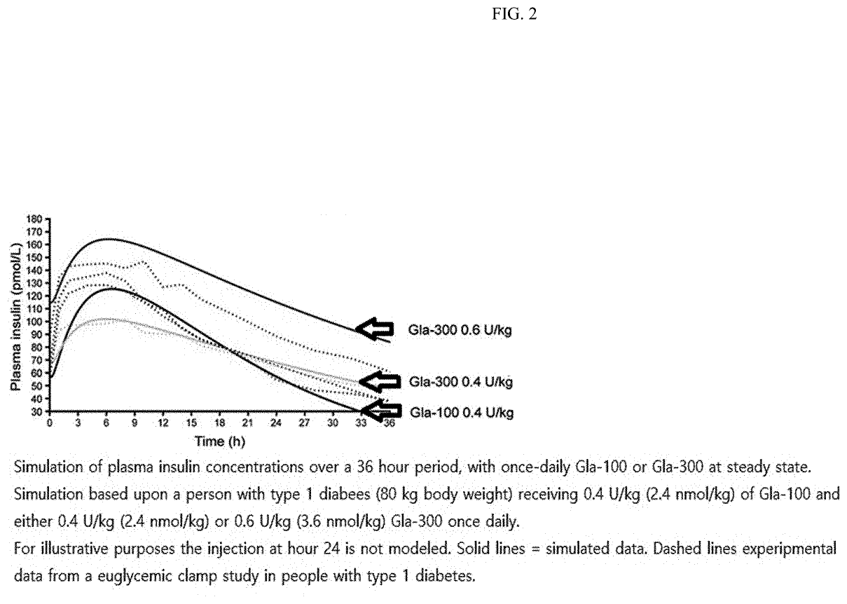

Insulin Glargine is clear unbuffered acidic solution and it forms precipitate when injected in Sc extracellular (ECM), physiological pH of 7.4 being its isoelectric point. After Insulin Glargine is injected, the Insulin Glargine subsequently dissociates into its hexamers, these hexamers then lose Zn and m-cresol to the surrounding fluid due to dilution and get converted to dimers and monomers. Insulin glargine monomer then gets metabolized to M1 and M2 in the blood, M1 being predominant metabolite. M1 interacts with insulin receptor of the target tissue and produces pharmacological effect (blood sugar lowering response) on the one hand and also gets degraded by insulinase to smaller peptides and then amino acids. According to the model Tojeo, 300 IU Insulin Glargine, forms more condensed precipitate and hence release of hexamers is relatively slower. Correspondingly, formation of Insulin Glargine dimers and monomers is also slower. Thus, absorption of the monomers and their conversion to M1, M2 is also proportionately slower as depicted in the FIG. 2 below.

However, no difference in plasma glargine/glargine metabolite pK profile was observed for the same dose of the drug administered by intravenous route.

Any in vitro model proposed should be able to mimic the in vivo model. An important observation that first phase of Insulin release upto Cmax is also proportionately slower for Insulin Glargine 300 IU/ml than its 100 IU/ml version at equal dose when delivered through Sc route.

We used 2 compartment in vitro model for in vivo validation of Sc Glargine pK, by testing formation of its precipitate in physiological buffered solution (“PBS”) in dialysis tubing representing Sc ECM. Redissolution of the precipitate involves dissociation of the precipitate to glargine hexamer, dimer and monomer in compartment 1 and formation of its metabolites M1, M2 followed by diffusion through 0.1 micron pores (representing pores of capillaries in the subcutaneous tissue) of dialysis membrane into the dialysate, 1 L PBS solution representing blood, Compartment 2. Thus, redissolution of insulin glargine precipitate in the dialysis tubing is compartment 1, while transport/diffusion of soluble insulin glargine monomer and its metabolites represents compartment 2. However, in the absence of any target tissue or its equivalent in the in vitro compartment 2 no degradation of the drug may be observed.

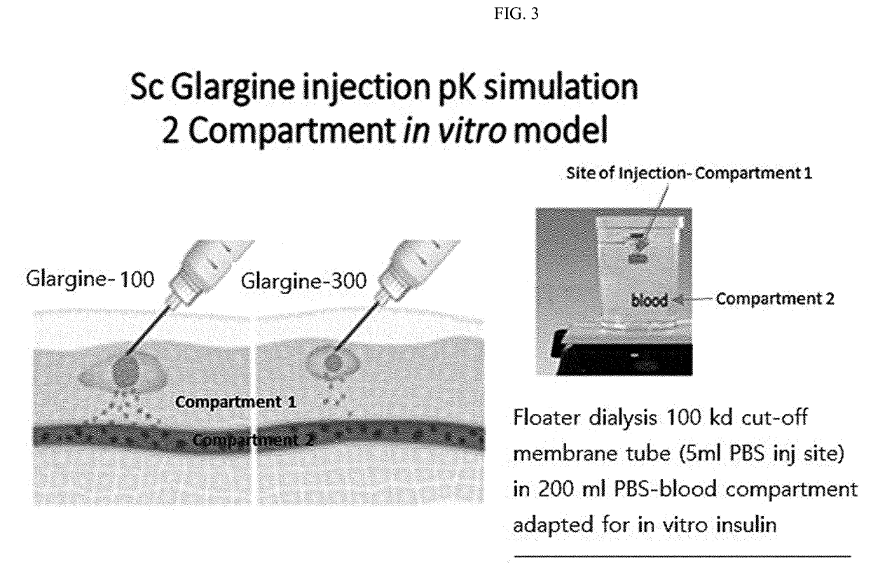

In case kinetics of Glargine 100 IU/ml (Example 1) and Glargine 300 IU/ml (Example 2) accumulation in the 1 L dialysate vessel display patterns as observed in vivo, the in vitro method stands validated and it can be used for testing regulated release of insulin/insulin glargine formulations. FIG. 3 depicts the proposed concept.

### Example 6: In-Vitro Kinetics of Protease Induced Release of Insulin Glargine

Two sets of in vitro insulin glargine equipment were set up as described above (example 5), Each equipment has a 10 ml stoppered 100 K_{d} cut off dialysis tube floating in a 1 L wide-mouthed stoppered Schott Bottle. Into thoroughly pre-washed and pre-sterilized (by dipping in 70% ethanol in water and equilibrating with PBS) dialysis tubing is added 5 ml sterile PBS, pH 7.4 and this tubing is fitted in the presterilized 1 L Schott bottle containing 1 L sterile PBS, pH 7.4 so that the dialysis tube remains vertical on floater. Sterile teflon coated 5.08 cm×0.95 cm magnet bar was placed in each bottle, the device was placed on magnetic stirrer under aseptic hood.

0.6 ml Lantus, 100 IU/ml glargine of Example 1, was added to PBS in the dialysis tubing of the first set while 0.2 ml Tojeo, 300 IU/ml glargine of Example 2, was added to the second.

To each 1^{st} compartment also added 10 milli units of filter sterilized r-rat trypsin, 20 micro liter solution. Magnetic stirrers were turned on and stirring was set at 100 rpm. 0.5 ml sample was withdrawn from each compartment (dialysis tubing as well as from the bottle) at different intervals. The samples were analyzed by HPLC for soluble insulin glargine and the result is tabulated below.

TABLE 5
Kinetics of in vitro protease induced Soluble
Insulin Glargine Release in 2 Compartments

| | Glargine (Milli IU/ml) Released | | | |
|---|---|---|---|---|
| | Lantus(Glargine 100 IU/ml; 0.6 ml) | | Tojeo(300 IU/ml; 0.20 ml) | |
| Time (h) | Compartment 1 | Compartment 2 | Compartment 1 | Compartment 2 |
| 0 | 0 | 0 | 0 | 0 |
| 0.50 | 90 | 89 | 75 | 66 |
| 1.00 | 118 | 120 | 124 | 126 |
| 2.00 | 230 | 228 | 155 | 153 |
| 4.00 | 288 | 290 | 194 | 196 |
| 6.00 | 360 | 362 | 248 | 246 |
| 8.00 | 450 | 460 | 310 | 307 |

From the result presented in table above, it appears that after 30 minutes insulin glargine release is similar in both compartments. Additionally, release of soluble Insulin Glargine is proportionately retarded in Tojeo (300 IU/ml Glargine formulation) than in Lantus (100 IU/ml Glargine formulation) due to more compact Insulin Glargine Precipitate/Crystal formation in compartment 1. Similar observation has been reported for in vivo human and animal pK studies.

Hence, we decided to use this in vitro method for evaluating the effect of RPC addition to Insulin Glargine Formulations for Glargine redissolution from the depot in compartment 1 and absorption/diffusion into compartment 2.

### Examples 7 and 8: Effect of Different Concentrations of Collagen on Insulin Glargine

Prepared five aliquots (2 times concentration) of 10 ml each, of insulin glargine 100 IU/ml formulation, in five 20 ml vials. And marked them with numbers 1 to 5. Number 1 was blank and 2 to 5 number vials received RPC 10, 20, 40 and 50 mg respectively (in 0.25, 0.5, 1.0 and 1.25 ml volume, respectively) in 2 times amounts. Made up the volume of each of the five vials to 20 ml (Final insulin glargine 100 IU/ml concentration).

Set up 5 in vitro insulin Glargine release devices (in vitro model). To the control set up injected 0.5 ml insulin glargine solution with RPC 0 mg/ml (100 IU/ml) in the compartment 1, the dialysis tubing. Similarly added 0.5 ml from glargine formulations containing 1 mg/ml RPC, 2 mg/ml RPC, 4 mg/ml RPC and 5 mg/ml RPC to the corresponding 1^{st} compartments of number 2, 3, 4, and 5 in vitro insulin glargine release devices, respectively. 0.5 ml samples were withdrawn at different time intervals from the compartment 2, the bottle, of each. The samples were analyzed by RPHPLC insulin glargine content.

TABLE 6
RPC Concentration Dependent retardation of Soluble
Insulin Glargine (100 IU/ml) Release

| | Milli IU Insulin Glargine Released | | | | |
|---|---|---|---|---|---|
| | RPC Concentration (mg/ml) | | | | |
| Time (h) | 0 | 1 | 2 | 4 | 5 |
| 0 | 0 | 0 | 0 | 0 | 0 |
| 0.5 | 80 | 78 | 65 | 58 | 60 |
| 1.0 | 175 | 170 | 80 | 98 | 120 |
| 2.0 | 230 | 220 | 160 | 145 | 160 |
| 4.0 | 288 | 260 | 250 | 195 | 190 |
| 6.0 | 360 | 348 | 376 | 247 | 255 |
| 8.0 | 455 | 440 | 400 | 305 | 310 |

Prepared five aliquots (two times concentration) of 10 ml each, of Insulin Glargine 300 IU/ml formulation, in five 20 ml vials. Marked them with numbers 1 to 5. Number 1 was blank and 2 to 5 number vials received RPC 10, 20, 40 and 50 mg each (in 0.25, 0.5, 1.0 and 1.25 ml volume) in 2 times amounts and made up the volume of each to 20 ml.

TABLE 7
RPC Concentration Dependent retardation of Soluble
Insulin Glargine (300 IU/ml) Release

| | Milli IU Insulin Glargine Released | | | | |
|---|---|---|---|---|---|
| | RPC Concentration (mg/ml) | | | | |
| Time (h) | 0 | 1 | 2 | 4 | 5 |
| 0 | 0 | 0 | 0 | 0 | 0 |
| 0.5 | 58 | 55 | 50 | 45 | 50 |
| 1.0 | 90 | 88 | 75 | 50 | 55 |
| 2.0 | 148 | 150 | 115 | 60 | 65 |
| 4.0 | 230 | 230 | 166 | 126 | 136 |
| 6.0 | 315 | 318 | 210 | 190 | 205 |
| 8.0 | 397 | 400 | 263 | 255 | 279 |

### Example 9: In-Vitro Release of Unit Composition Formula (100 IU/ml) of Example 1

TABLE 8
In-Vitro Release of Insulin Glargine-RPC (100 IU/ml) Composition

| | Glargine Release from Example 1 |
|---|---|
| Time (h) | (Milli IU/ml) |
| 2 | 148 |
| 4 | 200 |
| 6 | 258 |
| 8 | 318 |

### Example 10: In-Vitro Release of Unit Composition Formula (300 IU/ml) of Example 2

TABLE 9
In-Vitro Release of Insulin Glargine-RPC (300 IU/ml) Composition

| | Glargine Release from Example 2 |
|---|---|
| Time (h) | (Milli IU/ml) |
| 2 | 50 |
| 4 | 95 |
| 6 | 142 |
| 8 | 196 |

1. A stable ultra long acting injectable pharmaceutical composition comprising an insulin analogue or derivative having an isoelectric point between 5 and 8.5, or a physiologically tolerated salt thereof, collagen and one or more pharmaceutically acceptable excipients or carrier,
Wherein,
the pharmaceutical composition has pH between 2-5;
the pharmaceutical composition is a solution when administered and forms a depot upon administration at physiological pH; and
the pharmaceutical composition exhibits increased bioavailability and longer duration of action compared to the marketed composition comprising the said ‘insulin analogue or derivative having an isoelectric point between 5 and 8.5’ at same dose.

2. The stable ultra long acting injectable pharmaceutical composition as claimed in claim 1, wherein the ‘insulin analogue or derivative having an isoelectric point between 5 and 8.5, or a physiologically tolerated salt thereof is insulin glargine.

3. The stable ultra long acting injectable pharmaceutical composition as claimed in claim 2, wherein the insulin glargine is present in concentration range of 100 IU-1000 IU/ml.

4. The stable ultra long acting injectable pharmaceutical composition as claimed in claim 1, wherein the collagen is human collagen.

5. The stable ultra long acting injectable pharmaceutical composition as claimed in claim 1, wherein the duration of action is more than 24 h.

6. The stable ultra long acting injectable pharmaceutical composition as claimed in claim 1, wherein the pharmaceutical composition is administered two or three times in a week.

7. The stable ultra long acting injectable pharmaceutical composition as claimed in claim 1, wherein the pharmaceutical composition is less immunogenic compared to the marketed composition comprising the said ‘insulin analogue or derivative having an isoelectric point between 5 and 8.5, or a physiologically tolerated salt thereof at same dose.

8. The stable ultra long acting injectable pharmaceutical composition as claimed in claim 1, wherein the pharmaceutically acceptable excipients is selected from the group consisting of one stabilizers, isotonic agents, surfactants, buffers, preservatives, antioxidants, acidifying agents, pH modifying agents, organic solvents, aqueous solvents, solubilising agents and combination thereof.

9. A method for treating a metabolic disorder, the method comprising administering the stable ultra long acting injectable pharmaceutical composition according to claim 1 to a subject in need thereof.

10. The method of claim 9, wherein the metabolic disorder is one or more selected from the group consisting of insulin resistance syndrome, diabetes, hyperlipidemia, fatty liver disease, cachexia, obesity, atherosclerosis and arteriosclerosis.

11. A method for treating Type I or Type II Diabetes Mellitus in a patient comprising administering a pharmaceutical composition of claim 1 to a patient in need thereof.

12. A process for preparing a stable ultra long acting injectable solution comprising an insulin analogue or derivative having an isoelectric point between 5 and 8.5, or a physiologically tolerated salt thereof, collagen and one or more pharmaceutically acceptable excipients or carrier having pH between 2-5, wherein the process comprises
a) dissolving crystals of insulin glargine with HCl and water for injection;
b) preparing buffer having pH 2-5;
c) dissolving isotonic agent in buffer of step b);
d) diluting concentrated insulin glargine of step a) with the buffer of step c)
e) adding collagen the solution of step d) with constant stirring;
f) adjusting the final pH of the solution between 2-5.

13. The process for preparing a stable ultra long acting injectable solution of claim 12, wherein the ‘insulin analogue or derivative having an isoelectric point between 5 and 8.5, or a physiologically tolerated salt thereof is insulin glargine.

14. A method for modulating the release of an insulin analogue or derivative having an isoelectric point between 5 and 8.5, or a physiologically tolerated salt thereof from a pharmaceutical composition,
wherein the method comprises formulating a pharmaceutical composition comprising an an insulin analogue or derivative having an isoelectric point between 5 and 8.5, or a physiologically tolerated salt thereof, collagen and one or more pharmaceutically acceptable excipients or carrier at pH 2-5 and by precipitating the said composition at physiological pH upon administration.
